(19) United States
(12) Patent Application Publication
(10) Pub. No.: US 2012/0028246 A1
(43) Pub. Date: Feb. 2, 2012

(54) KIT FOR DETECTING HIGHLY PATHOGENIC AVIAN INFLUENZA VIRUS SUBTYPE H5N1

(75) Inventors: Takaaki Nakaya, Osaka (JP); Anariwa Du, Osaka (JP); Yusuke Shibai, Kanagawa (JP); Daisuke Itoh, Kanagawa (JP); Hisahiko Iwamoto, Kanagawa (JP)

(73) Assignees: TANAKA KIKINZOKU KOGYO K.K., Tokyo (JP); OSAKA UNIVERSITY, Suita-shi, Osaka (JP)

(21) Appl. No.: 13/255,793

(22) PCT Filed: Oct. 1, 2009
(86) PCT No.: PCT/JP2009/005065

§ 371 Date: Oct. 11, 2011

(30) Foreign Application Priority Data
Mar. 12, 2009 (JP) 2009-059544
Jun. 9, 2009 (JP) 2009-138714
Jul. 3, 2009 (JP) 2009-159020

(51) Int. Cl. C12Q 1/70

(52) U.S. Cl. 435/5

(57) ABSTRACT

Disclosed by the invention are an immunoassay kit and an immunoassay method for detecting highly pathogenic avian influenza virus subtype H5N1 rapidly, conveniently and specifically. Also disclosed are an immunochromatographic detection kit and an immunochromatographic detection method for detecting the virus subtype H5N1 rapidly, conveniently and specifically. It is found that a monoclonal antibody 4G6 produced by using the virus subtype H5N1 as an immunogen does not react with the subtype H5N2 virus or a subtype H5N3 virus and reacts only with a subtype H5N1 virus specifically. It is also found that only an avian influenza virus subtype H5N1 can be detected specifically by an immunoassay utilizing the monoclonal antibody 4G6. It is further found that the sensitivity of the detection of immunochromatography can be increased by adding a nonionic surface and a water-soluble vinyl polymer having a polar group containing an oxygen atom and a nitrogen atom to a developing solution to be used in the immunochromatography.

### TECHNICAL FIELD

The present invention relates to a kit for detecting highly pathogenic avian influenza virus subtype H5N1 and a detection method using the kit.

### BACKGROUND ART

Influenza is an infectious disease caused by influenza virus, targeting an organ such as nasopharynx, throat, bronchus, and the like. It is known to suddenly develop the symptoms such as pharyngalgia, runny nose, and cough as well as fever of 38° C. or higher, headache, joint pain, muscle pains, and the like. In Japan, there is a pattern that influenza starts to develop from the end of November to the beginning of December every year, and the number of the influenza increases around from January to March next year and decreases around April to May. As the influenza virus circulating among people in every year is completely adapted to humans, it almost has a relationship of coexistence. Thus, without a risk factor of pre-existing disease, advanced age, or the like, it is not so highly pathogenic to cause the death in most of infected people.

Influenza virus is classified into three types, A•B•C, depending on difference in antigenicity of a nuclear protein complex in the virus particle. It is believed that Type A influenza virus originated from a water fowl, in particular a duck. On the surface of Type A virus particle, glycoproteins including hemagglutinin (HA) and neuraminidase (NA) are present. HA has 16 subtypes and NA has 9 subtypes. From a duck, viruses, every possible combination of Type H1 to Type H16 of HA subtypes and Type N1 to Type N9 of NA subtypes, are detected (i.e., 144 kinds). These viruses infect other water fowls, domestic fowls, livestock, wild animals, and humans and keep causing an infection among the same animal species, thereby finally being adapted to each species. As a result of such adaptation, it is present as an influenza virus that is specific to each species. Influenza viruses that are currently circulating among humans (A/Russian type (H1N1) virus, A/Hong Kong type (H3N2) virus, and type B virus) are all considered to be viruses which have been originated from a water fowl and adapted to humans.

Due to annual epidemics, most people have immunity against common human influenza virus. Thus, even when a symptom is developed as a result of infection, most people easily recover though the fever may continue for several days. However, in case of an outbreak and epidemic of new influenza virus transmitted from bird to human, no human being has immunity against the virus. Thus, it is expected to have a large number of morbid patients and results in an increase in severe cases or fatality.

In particular, among the Type A avian influenza viruses, subtype H5N1 is known to exhibit high pathogenicity to cause human death in a case of an infection via a domestic fowl, even though it does not exhibit any pathogenicity in a duck that is an original host. As such, it is called highly pathogenic avian influenza virus. Recently, several cases of human infection that are caused by the influenza developed due to the virus infection of a domestic fowl with the virus subtype H5N1 are reported. Under these circumstances, there is great concern over the possibility that the avian influenza virus subtype H5N1 mutates so as to get easily transmitted from human to human and results in the transformation into a new influenza virus. Thus, for preventing an outbreak of new influenza, it is now a very important task to find out early the infection of a domestic fowl or a wild bird with the avian influenza virus, subtype H5N1 that is highly pathogenic and its transmission from bird to human and to deal with them appropriately.

Meanwhile, diagnosis of the infection with the avian influenza virus subtype H5N1 is currently carried out by identifying the H5 gene by RT-PCR after isolating the virus from a nasal or nasopharyngeal swab or a cloacal swab of specimen. However, as special devices and techniques are required for this diagnostic method, a fast and convenient diagnosis cannot be made in a chicken nursery or an outdoor environment where the infection of the virus is suspected. For the infection with a common human influenza virus, a rapid diagnosis kit based on immunochromatography to detect an influenza virus antigen at early stage of the development has been already used. However, such kit is to distinguish the influenza virus infection from the infections with other viruses or bacteria, and it detects a nuclear protein and the like as an antigen which has relatively little mutation among the proteins of the influenza virus. As such, although the influenza Type A or Type B virus can be identified, it cannot identify the subtypes based on hemagglutinin (HA) and neuraminidase (NA) which often change the antigenicity in the same subtype.

In order to identify the subtype of the type A influenza virus, it is proposed that a monoclonal antibody against HA in the virus subtype H5 is produced and the measurement is carried out based on immunochromatography assay (Patent Documents 1, and 2). However, the monoclonal antibody used for the measurements broadly recognizes virus subtype H5 which has HA of subtype H5. It is also reported that, in 2009 a monoclonal antibody against the influenza virus subtype H5N1 was established by having the virus subtype H5N1 isolated from a crow in Kyoto, Japan as an immunogen (Patent Document 3 and Non-patent Document 1). However, some of these monoclonal antibodies (3C11, 4C12, 3H12, and 3H4) broadly recognize HA of subtype H5 including the subtype H5N1 (Patent Document 3). As a result, the immunoassay kit using these antibodies detect not only the subtype H5N1 but also a low pathogenic avian influenza virus of subtypes H5N2, H5N3, and the like, and no rapid diagnosis kit has been provided for specific detection of the highly pathogenic avian influenza virus subtype H5N1. Because the virus subtype H5N1 has high pathogenicity and high mortality, there has been a strong need for development of a rapid diagnosis kit for specific detection of the virus subtype H5N1.

### PRIOR ART DOCUMENTS

### Patent Document

Patent Document 1: Japanese Patent Application Laid-open (JP-A) No. 2007-261988
Patent Document 2: JP-A No. 2008-196967
Patent Document 3: JP-A No. 2008-104450

### NON-PATENT DOCUMENT

Non-patent Document 1: Biochem Biophys Res Commun. 2009 Jan. 9; 378(2): 197-202

### SUMMARY OF THE INVENTION

### Problems to be Solved by the Invention

The invention provides an immunoassay kit and an immunoassay method for detecting highly pathogenic avian influenza virus subtype H5N1 rapidly, conveniently and specifically. Also provided by the invention are an immunochromatographic detection kit and an immunochromatographic detection method for specifically detecting the virus subtype H5N1 rapidly, conveniently and with high sensitivity.

### Means for Solving the Problems

As a result of intensive Studies, inventors of the invention found that a monoclonal antibody 4G6 produced by using the virus subtype H5N1 as an immunogen does not react with a subtype H5N2 virus or a subtype H5N3 virus and reacts specifically with a virus subtype H5N1. It is also found out by the inventors that only the avian influenza virus subtype H5N1 can be detected specifically by an immunoassay utilizing the monoclonal antibody 4G6. It is further found out by the inventors that the sensitivity of detection can be enhanced by adding a nonionic surface active agent and a water-soluble vinyl polymer having a polar group containing an oxygen atom and a nitrogen atom to a developing solution to be used in the immunochromatography.

That is, the invention relates to an immunoassay kit and an immunoassay method for specifically detecting highly pathogenic avian influenza virus subtype H5N1. The invention also relates to an immunochromatographic detection kit and an immunochromatographic detection method for specifically detecting the influenza virus subtype H5N1 with high sensitivity.

Herein below, the invention is explained in greater detail.

(1) A detection kit for type A influenza virus subtype H5N1, for detecting a substance of interest in sample by immunochromatography assay, including a chromatography medium containing a first reagent at decision part, a labeling agent in which a second reagent is conjugated to a labeling substance, and a developing solution; containing one or both of the first reagent and the second reagent being an antibody specifically recognizing type A influenza virus subtype H5N1.
(2) The detection kit according to (1), wherein the first reagent specifically recognizes type A influenza virus subtype H5N1.
(3) The detection kit according to (1) or (2), wherein the antibody specifically recognizing type A influenza virus subtype H5N1 is a monoclonal antibody recognizing a conformational epitope containing an asparaginic acid that is the 59^{th} amino acid of hemagglutinin in the virus subtype H5N1.
(4) The detection kit according to (3), wherein the monoclonal antibody recognizing the conformational epitope containing an asparaginic acid that is the 59^{th} amino acid of hemagglutinin in the virus subtype H5N1, is a monoclonal antibody produced by a mouse-mouse hybridoma 4G6 (deposit number: FERN BP-11130).
(5) The detection kit according to any one of (1) to (4), wherein the remaining first reagent or the second reagent is a monoclonal antibody recognizing a consecutive epitope presenting in the region of 273-342aa in hemagglutinin HA1 domain of influenza virus subtype H5N1.
(6) The detection kit according to (5), wherein the monoclonal antibody recognizing a consecutive epitope presenting in the region of 273-342aa in hemagglutinin HA1 domain of influenza virus subtype H5N1 is a monoclonal antibody produced by a mouse-mouse hybridoma 3H4 (deposit number: FERM BP-11173) or a mouse-mouse hybridoma 3H12 (deposit number: FERN BP-11174).
(7) The detection kit according to any one of (1) to (6), wherein the developing solution contains a non-ionic surface active agent with HLB value of from 13 to 18.
(8) The detection kit according to (7), wherein a concentration of the non-ionic surface active agent is 0.1 to 1.0%.
(9) The detection kit according to any one of (1) to (8), wherein the developing solution further contains a vinyl-based water soluble polymer having a polar group with an oxygen atom and a nitrogen atom.
(10) The detection kit according to (9), wherein a concentration of the vinyl-based water soluble polymer is 0.5 to 2.0%.
(11) The detection kit according to (9) or (10), wherein the vinyl-based water soluble polymer is polyvinyl pyrrolidone.
(12) The detection kit according to any one of (7) to (11), wherein the labeling substance is an insoluble carrier.
(13) The detection kit according to (12), wherein the insoluble carrier is a colloidal gold particle.
(14) A method of detecting type A influenza virus subtype H5N1 in a sample by using the detection kit according to any one of (1) to (13), containing processes of bringing a sample into contact with a chromatography medium, bringing a labeling agent into contact with the chromatography medium together with or subsequent to the sample, and developing the sample and the labeling agent with an aid of a developing solution.

### Effects of the Invention

The immunoassay kit of the invention can detect only the highly pathogenic avian influenza virus subtype H5N1 rapidly, conveniently, and specifically without showing any cross reaction with the subtype H5N2 or the subtype H5N3 which has low pathogenicity. Further, the immunochromatographic detection kit of the invention can detect the virus subtype H5N1 rapidly, conveniently, and specifically with a detection sensitivity that is practically usable.

### BRIEF DESCRIPTION OF THE DRAWINGS

FIG. 1 is a graph of comparison in the coloration strength that is measured by an immunochromato reader at a decision part, wherein various development solutions having different composition are used for immunochromatographic detection of HA recombinant protein (from ABR) of the virus subtype H5N1 at the concentration of 2 ng/mL using the detection kit of the invention. The composition of the development solutions A to H is described in Table 5. When the value measured by immunochromato reader (manufactured by Hamamatsu Photonics K.K.) is 20.0 or more, color development can be clearly identified with naked eye.

FIG. 2 is a graph of the coloration strength obtained at a decision part when the virus subtype H5N1, the virus subtype H5N2, the virus subtype H5N3, and the virus subtype H1N1 are detected by using the detection kit of the invention. The composition of the development solution is described in Table 5. The virus subtype H5N1 showed, even at the concentration of 10⁴ pfu/mL, a measurement value of 15.0 or more which is a critical value for naked eye measurement using an immunochromato reader manufactured by Tanaka Kikinzoku Kogyo K.K. Meanwhile, the measurement value obtained from the virus subtype H5N2, the virus subtype H5N3, and the virus subtype H1N1 was 15.0 or less at the concentration of 10⁶ pfu/mL, and the color development cannot be confirmed with naked eye.

FIG. 3 is a graph of the coloration strength obtained at a decision part when the influenza virus strains having various hemagglutinin (HA) subtypes and neuraminidase (NA) subtypes are detected by using the detection kit of the invention. With the detection kit of the invention, color development due to the cross reaction was not shown from the virus strains other than the virus subtype H5N1 at the concentration of 10⁵ pfu/mL.

### MODES FOR CARRYING OUT THE INVENTION

The detection kit of the invention includes a chromatography medium which has the first reagent at a decision part, a labeling agent in which the second reagent is conjugated to a labeling substance, and a developing solution. The detection kit of the invention allows specific detection of the highly pathogenic avian influenza virus H5N1 (herein below, also referred to as AIV H5N1) as a substance of interest in a sample based on the immunochromatographic principle by which a substance of interest is detected using a specific binding reaction between an antigen and an antibody therefor.

Raw materials of the chromatography medium of the invention are not specifically limited, as long as they are an inert microporous material showing capillary phenomena and having no reactivity with a labeling agent, a substance of interest, and the like. Specific examples include a fibrous or non-woven fibrous matrix and a membrane that are composed of polyurethane, polyester, polyethylene, polyvinyl chloride, polyvinylidene fluoride, nylon, or cellulose derivatives such as nitrocellulose and cellulose acetate; a filter paper; a glass fiber filter; a cloth; cotton; and the like. Preferred examples include a membrane composed of cellulose derivatives or nylon, a filter paper, a glass fiber filter, and the like. More preferred examples include a nitrocellulose membrane, a membrane of mixed nitrocellulose-ester (i.e., mixture of nitrocellulose and cellulose acetate), a nylon membrane, and a filter paper.

The shape and size of the chromatography medium is not specifically limited and they are only required to be suitable in terms of actual operation and observation of results. For more convenient operation, a support made of plastics and the like may be provided on the back side of the chromatography medium which has a decision part on its surface. Properties of the support are not specifically limited. However, when measurement results are observed with naked eye, it is preferable that the support has a color that is different from the color exhibited by a labeling agent. In general, a colorless or white colored support is preferable.

The chromatography medium optionally contains a sample addition part (i.e., a sample pad and the like) for adding a sample including a substance of interest, a part for removing solid components from a sample (i.e., a solid component removing part and the like), a developing solution addition part for adding a developing solution, an absorption part for absorbing a labeling agent not captured in the decision part or a developing solution (i.e., absorption pad and the like), a comparison part for ensuring normal measurement, and the like. Members for the above parts are not specifically limited as long as they allow the migration of a sample solution or a developing solution based on capillary phenomena. They are generally selected from various porous materials such as a nitrocellulose membrane, a filter paper, and a glass fiber filter depending on purpose. They are arranged to be connected to the chromatography medium immobilized with the first reagent via capillary tube.

According to the invention, the decision part is formed by immobilization of the first reagent onto the chromatography medium. As for a method of immobilizing the first reagent onto the chromatography medium, there is a direct immobilization method by which the first reagent is immobilized directly onto the chromatography medium via physical or chemical means and an indirect immobilization method by which the first reagent is physically or chemically linked to fine particles such as latex particles, and the fine particles are fixed on the chromatography medium for immobilization.

With respect to the direct immobilization method, it may be achieved by physical adsorption or covalent bond. When the chromatography medium is a nitrocellulose membrane or a mixed nitrocellulose ester membrane, physical adsorption can be generally carried out. For covalent bond, cyanobromide, glutaraldehyde, carbodiimide, and the like are generally used for activation of the chromatography medium, and any method can be used. The indirect immobilization method includes a method of linking the first reagent to insoluble fine particles and then immobilizing them onto the chromatography medium. As for the particle size of insoluble fine particles, the size which allows fixation onto the chromatography medium with no migration can be selected. Preferably, it is a fine particle having the average particle diameter of 10 μm or less. There are many particles already known to be used for an antigen and antibody reaction. From the viewpoint of easy control of sensitivity and the like, in the invention, the direct immobilization is preferable. For immobilization of the first reagent to the chromatography medium, various methods can be employed and examples include various techniques using a micro syringe, a pen equipped with regulatory pump, ink spray printing, and the like. The shape of the decision part is not specifically limited. Immobilization can be made on a circular spot, a line which extends along the direction perpendicular to the development of the chromatography medium, a number, a letter, or a symbol such as + and −, and the like.

Further, if necessary, the chromatography medium obtained after immobilization of the first reagent can be subjected to a blocking treatment. Examples of the blocking agent which can be used for blocking treatment include commercially available blocking agents such as Blocking Peptide Fragment (manufactured by Toyobo Co., Ltd.) and hydrophilic macromolecular polymer as well as proteins such as bovine serum albumin, skim milk, casein, and gelatin.

The labeling agent of the invention is composed by linking the second reagent to a labeling substance. As the labeling substance, an enzyme or an insoluble carrier can be used. Examples of the enzyme include alkaline phosphatase, horseradish peroxidase, β-galactosidase, urease, glucose oxidase, and the like, and they can be used with a known color-developing substrate corresponding to each enzyme. Examples of the insoluble carrier include colloidal metal particles such as gold, silver, and platinum, colloidal metal oxide particles such as iron oxide, colloidal non-metal particles such as sulfur, latex particles made of synthetic macromolecules, and others. Examples of the colloidal metal particles and colloidal metal oxide particles include colloidal gold particles, colloidal silver particles, colloidal platinum particles, colloidal iron oxide particles, colloidal aluminum hydroxide particles, and the like. In particular, colloidal gold particles and colloidal silver particles are preferable in terms of appropriate particle size. Colloidal gold particles and colloidal silver particles are preferable in that they show red color and yellow color, respectively. The average particle diameter of these colloidal metal particles is within the range of 1 nm to 500 nm. The range of 10 nm to 150 nm which gives strong color development is preferable. More preferably, it is within the range of 40 nm to 100 nm. Examples of the latex particles include a copolymer of styrene and methacrylic acid, a copolymer of styrene and itaconic acid, and the like. The average particle diameter of the latex particles is preferably within the range of 50 nm to 500 nm. The labeling substance used as a labeling agent of the invention is preferably an insoluble carrier. More preferably, it is colloidal metal particles. Still more preferably, it is colloidal gold particles.

As for the colloidal metal particles, when the colloidal gold particles are used, for example, commercially available ones can be used. Alternatively, colloidal gold particles can be prepared according to a method known in the art, for example, reducing chloroauric acid with sodium citrate.

As for the method of labeling the second reagent used in the invention with a labeling substance, it can be carried out according to a method known in the art, for example, physical adsorption, chemical bonding, or the like. For example, when the second reagent is labeled with colloidal gold particles, the production is carried out by adding an antibody which is the second reagent, for physical adsorption to a solution in which gold particles are dispersed in a colloidal state, and then adding a solution of bovine serum albumin, a commercially available blocking agent described above, or the like for blocking the particle surface wherein an antibody is not linked.

The antibody which can be used as the first or second reagent of the invention is an antibody which binds to a substance of interest, i.e., the avian influenza virus subtype H5N1. Either a polyclonal antibody or a monoclonal antibody may be used as long as the antibody has such property. Preferably, a monoclonal antibody is used for any one of the first reagent and the second reagent. More preferably, a monoclonal antibody is used for both the first reagent and the second reagent. For immobilization to the chromatography medium or conjugation to a labeling substance, these antibodies may also be used as a fragment such as Fab or F(ab′)₂ having a binding ability. When the antibody used as the first or second reagent of the invention can bind to AIV H5N1, it is possible to detect the presence of AIV H5N1 on a decision part of the chromatography medium. However, for the specific detection of the highly pathogenic avian influenza virus subtype H5N1 without having any cross reaction with other virus subtypes such as low pathogenic virus subtype H5N2 or H5N3, it is preferable to use an antibody which specifically recognizes the avian influenza virus subtype H5N1 as either one or both of the first reagent and the second reagent.

The antibody which specifically recognizes the type A influenza virus subtype H5N1 indicates an antibody which reacts with AIV H5N1 antigen such as AIV H5N1., a cell infected with AIV H5N1, or the hemagglutinin protein of AIV H5N1 but does not show any reactivity toward an antigen of other virus subtypes such as AIV H5N2 and AIV H5N3, according to immunofluorescence assay (IFA), Western blotting, or the like. If an antibody shows the specific reactivity only to AIV H5N1 at the relatively low concentration according to an experiment for comparing the binding ability toward AIV H5N1 antigen and the binding ability toward other virus subtypes, it can be suitably used as an antibody of the invention. It is more preferable to use an antibody which recognizes an epitope or a conformational epitope consisting of consecutive amino acid sequences that are only found in AIV H5N1. Examples of the epitope which has been confirmed to be present specifically in AIV H5N1 include the conformational epitope wherein an asparaginic acid which is the 59^{th} amino acid of hemagglutinin as a surface protein of AIV H5N1, is contained. The Asp at position 59 in HA is highly preserved among the AIV H5N1 strains that are widely circulated from the year of 2003 until now (1813 strains/1870 strains). Meanwhile, from AIV H5N2 and AIV H5N3, no virus is known to have Asp at position 59 in HA. Thus, such antibody of the invention, which recognizes an epitope that is confirmed to be present specifically in AIV H5N1, is preferable. Specific examples of the antibody include the monoclonal antibody 4G6 produced by a mouse-mouse hybridoma 4G6 (deposit number: FERM BP-11130)

With regard to the AIV H5N1-specific antibody used in the invention, it is not intended to limit a source of the antibody or, a method of producing the antibody as long as it specifically recognizes the AIV H5N1 antigen. Further, as long as it has a specific reactivity toward the AIV H5N1-specific epitope, a fragment such as Fab, Fab′, F(ab′)₂, and Fv can also be used, and also the CDR and FR parts in the variable region of the antibody may have a different origin.

The antibody which specifically recognizes the type A influenza virus subtype H5N1 used in the invention can be produced by administering, as an immunogen, AIV H5N1 inactivated by para formaldehyde and the like, cells such as MDCK cells infected with AIV H5N1, transformed cells in which HA gene originating from AIV H5N1 is expressed, HA protein purified from AIV H5N1, its recombinant protein, or the like, to a known immunized animal such as mouse and rabbit. When an antibody which recognizes a conformational epitope containing an asparaginic acid as the 59^{th} amino acid of HA originating from AIV H5N1, which is a suitable antibody of the invention, is to be produced, in addition to the immonogens described above, transformed cells which express chimeric HA consisting of H5N1 HA including Asp59 of H5N1 HA and HA originating from other virus subtypes, or transformed cells which express H5N1 HA in which one or several amino acids are deleted, substituted, or added may be also used as an immunogen. Further, by screening a commercially available peptide library kit based on phage display and the like with the antibody 4G6, a peptide mimicking the epitope of 4G6 can be obtained, and it can also be used as an immunogen.

When the antibody is obtained in the form of a monoclonal antibody, spleen cells are collected from the immunized animal to which an immunogen is administered, and the cells are fused with myeloma cells by a standard method known in the art to prepare an antibody-producing hybridoma. Alternatively, by screening the library of the antibody gene with AIV H5N1 antigen, the monoclonal antibody can be obtained without preparing any hybridoma. In the library used for obtaining a monoclonal antibody, an antibody protein and a gene which encodes the antibody protein show one-to-one relationship according to a display technique, and therefore a gene for desired antibody can be immediately obtained by screening against the target antigen. Representative examples of the display technique include a phage display. However, in addition to a method of using cells of yeast display, bacteria display, and the like, a method of using cell-free translation system of cDNA display, mRNA display, ribosome display, and the like is also known.

As for the method of screening an antibody which specifically recognizes the type A influenza virus subtype H5N1, there is a method of selecting an antibody showing stronger reactivity to AIV H5N1 antigen compared to other virus subtypes by immunofluorescence assay (IFA), Western blotting, and the like by using AIV H5N1, cells infected with AIV H5N1, HA of AIV H5N1, or the like as an antigen. In particular, for screening an antibody which recognizes a conformational epitope containing Asp59 of H5N1 HA, the antibody can be selected by using AIV H5N1, cells infected with AIV H5N1, or the like, in which the stereo structure of HA protein is preserved, as an antigen, and using as an indicator, the activity of culture supernatant of hybridoma or member of the library for inhibiting the binding between the antibody 4G6 and the antigen. As for another screening method, by using transformed cells which express HA originating from H5N1 and another transformed cell which expresses H5N1 HA variant in which Asp59 of the HA is substituted with another amino acid, the antibody can be screened while using the specific reactivity toward the cells expressing H5N1 HA with Asp59 as an indicator.

The antibody 4G6, which is one of the monoclonal antibodies recognizing an AIV H5N1-specific epitope, was produced as follows. Specifically, A/crow/Kyoto/53/2004 H5N1 virus was purified by ultracentrifuge (25,000 rpm, 1 hour) with 20% sucrose cushion and fixed with 4% para formaldehyde. As an antigen, the resultant was administered to a female BALB/c mouse together with Freund's complete adjuvant for initial immunization (2×10⁷ TCID₅₀/mouse). Two weeks later, the mouse was subjected to booster immunization with inactivated virions free of the adjuvant. Three days after the second booster immunization, the spleen cells were collected from the immunized mouse and fused with PAI myeloma cells by applying a standard method known in the art. Ten to fifteen days later, a hybridoma clone which produces an antibody was selected by immunofluorescence assay (IFA) which uses MDCK cells infected with AIV H5N1 as an antigen. The antibody 4G6 recognized the MDCK cells infected with H5N1 but did not recognize the MDCK cells infected with virus H5N2 or virus H5N3. To confirm that the antibody 4G6 recognizes the HA protein originating from the virus subtype H5N1, HA gene amplified by using PCR from virus H5N1 of not only clade 2.5 (A/crow/Kyoto/53/2004) but also clade 1 (A/Thailand/Kan353/2004) was cloned in pPoll plasmid, and used for transfection of 293T cells, together with PB2, PB1, PA and NP genes cloned in the expression plasmid pCAGGS. As a result, H5N1-HA expressing cells were produced. The antibody 4G6 recognized every 293T cells which express H5N1-HA.

In order to analyze more specifically the epitope recognized by the antibody 4G6, six chimeric pPolI-HA plasmids originating from H5N1-HA and H5N3-HA were constructed by exchanging the domains of 1-86 aa, 1-194 aa, or 1-340 aa in H5N1-HA and H5N3-HA. 293T cells were transfected with a series of pPolI-chimeric HA plasmid together with pCAGGS-PB2, -PB1, -PA, and -NP. After fixing, the cells were used as an antigen for IFA. The antibody 4G6 recognized the 293T cells in which H5N1-HA (1-86 aa)-H5N3-HA (87-567 aa) chimera HA, H5N1-HA (1-194 aa)-H5N3-HA (195-567 aa) chimera HA, and H5N1-HA (1-340 aa)-H5N3-HA (341-567 aa) chimera HA are expressed. However, it did not recognize the 293T cells in which H5N3-HA (1-86 aa)-H5N1-HA (87-567 aa) chimera HA, H5N3-HA (1-194 aa)-H5N1-HA (195-567 aa) chimera HA, and H5N3-HA (1-340 aa)-H5N1-HA. (341-567 aa) chimera HA are expressed. Specifically, the antibody 4G6 binds to the conformational epitope of 1-86 aa region of HA originating from H5N1. Sequence alignment of presumed amino acids in 1-86 aa region for each HA of A/crow/Kyoto/53/2004 (H5N1), A/Thailand/Kan353/2004 (H5N1), A/Duck/Hong Kong/342/78 (H5N2) and A/Duck/Hong Kong/820/80 (H5N3) was carried out. As a result, three positions in H5N1 HA were found to be different from the amino acids of H5N2 HA and H5N3 HA (i.e., position 51, 59 and 61). Thus, single amino acid-substituted mutant HA protein (i.e., K51R, D59S, or D61N) in which each of the amino acids of the subtype H5N1 at these positions are substituted with the corresponding amino acids in the subtype H5N2 or H5N3 was prepared, and then expressed in 293T cells. As a result of performing IFA with the expressed cells, it was found that the antibody 4G6 recognizes the mutant in which Lys at position 51 of H5N1 HA is substituted with Arg and the mutant in which Asp at position 61 is substituted with Asn. However, it did not recognize the mutant in which Asp at position 59 of H5N1 HA is substituted with Ser. Accordingly, it was found that the antibody 4G6 is an antibody which recognizes a conformational epitope including the asparaginic acid that is the 59^{th} amino acid of HA in the virus subtype H5N1.

The mouse-mouse hybridoma 4G6 producing the monoclonal antibody 4G6 was deposited with Patent Organism Depository Center of National Institute of Advanced Industrial Science and Technology [1-1-1, Higashi, Tsukuba, Ibaraki, Japan, Central 6 (zip code: 305-8566)] with deposit number of FERM BP-11130 and deposit date of May 21, 2009.

The antibody which specifically recognizes the type A influenza virus subtype H5N1 can be used for a known immunoassay such as a coagulation method, a radioimmunoassay, an enzyme immunoassay, and an immunochromatography assay, which specifically detect the AIV H5N1. In particular, according to immunochromatography assay, it can be suitably used as either one or both of the first reagent which constitutes the decision part of the chromatography medium and the second reagent which constitutes the labeling agent, respectively. More preferably, for efficient capturing of AIV H5N1, that is a substance of interest, on the decision part of the chromatography medium, the antibody specifically recognizing AIV H5N1 can be used as the first reagent.

When the antibody specifically recognizing AIV H5N1 is used as the first reagent or the second reagent of immunochromatography assay, the remaining first reagent or second reagent can be any one which binds to AIV H5N1 and an antibody exhibiting reactivity toward other virus subtypes can also be used. Examples of the preferred antibody include an antibody which does not inhibit the binding between AIV H5N1 in a substance of interest and an antibody which specifically recognizes AIV H5N1. Examples of the more preferred antibody include an antibody which can recognize an epitope different from the one recognized by the AIV H5N1-specific antibody. Examples of such antibody include an antibody which recognizes the consecutive amino acid sequence presenting in the region of 273-342aa in HA1 domain of AIV H5N1 hemagglutinin and the like.

Antibody binding to AIV H5N1 can be produced according to a standard method known in the art by using the influenza virus or virus-infected cells having the subtype H5 as an immunogen. Further, the antibody which recognizes the consecutive amino acid sequence present in the region of 273-342aa in HA1 domain of AIV H5N1 hemagglutinin can be prepared as an antibody produced from the mouse-mouse hybridoma 3C11, 4C12, 3H4, and 3H12 (Patent Document 3). Of these hybridomas, 3C11 and 4C12 have been deposited with Patent Organism Depository Center of National Institute of Advanced Industrial Science and Technology with deposit number of FERM P-21027 and FERN P-21028, respectively. Further, 3H4 and 3H12 have been deposited with Patent Organism Depository Center of National Institute of Advanced Industrial Science and Technology [1-1-1, Higashi, Tsukuba, Ibaraki, Japan, Central 6 (zip code: 305-8566)] with deposit number of FERM P-21029 and FERM P-21030, respectively, and deposit date of Sep. 20, 2006. Further, on Aug. 20, 2009, they were transferred to International Depository Organization with deposit number of FERM BP-11173 and FERM BP-11174, respectively.

The developing solution of the invention constitutes a mobile phase of immunochromatography assay. According to immunochromatography assay, by applying the principle of chromatography, a labeling agent bound to a substance of interest and a non-bound labeling agent are separated from each other by using a system including a stationary phase which can capture the substance of interest and a mobile phase which continuously flows while being in contact with the stationary phase. The developing solution is used for moving (or, developing) the substance of interest and the labeling agent through the inside of a chromatography medium that is made of microporous materials exhibiting capillary phenomena.

It is preferable that the developing solution of the invention generally contains water as a solvent and buffer agents such as phosphate salt, trishydroxymethyl aminomethane hydrochloric acid salt, HEPES, and Good's buffer agent, and inorganic salts such as sodium chloride. Further, if necessary, it may contain a protein component such as bovine serum albumin (BSA) (the content is generally from 0.01% by weight to 10% by weight), preservatives, and the like. The developing solution used for the invention further contains a surface active agent. More preferably, it further contains a vinyl-based water soluble polymer having a polar group containing an oxygen atom, as represented by polyvinyl pyrrolidone.

As for the non-ionic surface active agent added to the developing solution, a polyoxyethylene-based surface active agent, having the HLB value of preferably 10 to 18, and more preferably 13 to 18, can be used. Examples of suitable polyoxyethylene-based surface active agent include polyoxyethylene alkyl ether, polyoxyethylene sorbitan fatty acid ester (trade name “Tween” series), polyoxyethylene p-t-octylphenyl ether (trade name “Triton” series), polyoxyethylene p-t-nonylphenyl ether (trade name “Triton N” series), and the like. More specifically, “Tween” series particularly include Tween20 (trade name) (HLB value: 16.7), Tween40 (trade name) (HLB value: 15.6), Tween60 (trade name) (HLB value: 15.0), and Tween80 (trade name) (HLB value: 14.9). “Triton” series particularly include Triton X-100 (trade name) (HLB value: 13.5), Nonidet P-40 (trade name) (HLB value: 13.1), Triton X-102 (trade name) (HLB value: 14.6), Triton X-165 (trade name) (HLB value: 15.8), and Triton X-405 (trade name) (HLB value: 17.9). “Triton N” series particularly include Triton N-101 (trade name) (HLB value: 13.5), Triton N-111 (trade name) (HLB value: 13.8), and Triton N-150 (trade name) (HLB value: 15.0). The non-ionic surface active agent may be used either singly or in combination of two or more. The content of the non-ionic surface active agent described above is not specifically limited. However, it is in the range of 0.01 to 10.0% by weight compared to the total weight of the developing solution. Preferably, it is in the range of 0.1 to 5.0% by weight, more preferably in the range of 0.1 to 1.0% by weight, and still more preferably in the range of 0.3 to 1.0% by weight compared to the total weight of the developing solution.

As for the vinyl-based water soluble polymer which is further added to the developing solution, a vinyl-based water soluble polymer having a polar group containing an oxygen atom is preferable. Examples thereof include a polymer having a structural unit in which the double bond of the vinyl-based water soluble monomer having a polar group containing an oxygen atom is cleaved, for example, vinyl alcohol, vinyl methyl ether, (meth)acrylic acid, hydroxyalkyl(meth)acrylate, (meth)acrylamide, dimethyl(meth)acrylamide, vinyl pyrrolidone, and the like. More preferred examples include a vinyl-based water soluble monomer having a polar group containing an oxygen atom and a nitrogen atom. Still more preferred example include a polymer having a structural unit in which the double bond of a vinyl-based, non-ionic water soluble monomer having a polar group containing an oxygen atom, and a vinyl-based, non-ionic water soluble monomer having a polar group containing an oxygen atom and a nitrogen atom, is cleaved. Most preferred examples include a polymer having a structural unit in which the double bond of vinyl pyrrolidone is cleaved.

As for the vinyl-based water soluble polymer, a copolymer in which other vinyl-based monomer such as vinyl acetate and alkyl(meth)acrylate is copolymerized to the extent that the effect of the invention is not impaired, for example, 50 mol % or less, preferably 30 mol % or less, and particularly preferably 15 mol % or less, can be used.

Specific examples of the preferred include polyvinyl pyrrolidone (herein below, also referred to as PVP), dimethylacrylamide/vinyl pyrrolidone copolymer (copolymerization ratio of dimethyl acrylamide is 50 mol % or less), vinyl alcohol/vinyl pyrrolidone copolymer (copolymerization ratio of vinyl alcohol is 50 mol % or less), vinyl acetate/vinyl pyrrolidone copolymer (copolymerization ratio of vinyl acetate is 20 mol % or less), and the like.

The molecular weight of the vinyl-based water soluble polymer is generally 10,000 to 1,000,000, preferably 100,000 to 1,000,000, and more preferably 200,000 to 500,000. Further, the concentration of the vinyl-based water soluble polymer is, compared to the total weight of the developing solution, preferably 0.01 to 5.0% by weight, more preferably 0.1 to 3.0% by weight, and still more preferably 0.5 to 2.0% by weight.

In the patent application mentioned above (JP-A No. 2008-182630), relationship between the composition of the developing solution and the measurement sensitivity of immunochromatography assay is examined in detail. The application is incorporated in its entirety by reference in the specification of this application.

In the specification of the application mentioned above, determination is made on immunochromatography assay for detecting a nuclear protein of influenza virus or human hemoglobin protein as a substance of interest. When Tween20 as a non-ionic surface active agent is added to a developing solution, the measurement sensitivity was improved. Further, when PVP which is a vinyl-based water soluble polymer having a polar group containing an oxygen atom, is added to a developing solution, the effect is enhanced more. Meanwhile, when sodium cholate which is an ionic surface active agent, is added to a developing solution, no specific increase in measurement sensitivity was shown. Further, when sodium carboxymethyl cellulose (CMC•Na) or polyethylene glycol (PEG) is used as a water soluble polymer, there was a significant non-specific reaction in a negative sample which does not contain any substance of interest. The effect exhibited by the non-ionic surface active agent and the vinyl-based water soluble polymer having a polar group containing an oxygen atom that are added to the developing solution was also shown in the immunochromatography assay wherein the substance of interest is human hemoglobin protein. By adding a non-ionic surface active agent and a vinyl-based water soluble polymer having a polar group containing an oxygen atom represented by PVP to the developing solution, strength of coloration (i.e., signal) at the decision part is increased for the positive sample containing the substance of interest. However, when a negative sample containing no substance of interest is measured, strength of coloration (i.e., noise) was decreased so that a good signal to noise ratio can be obtained. When the vinyl-based water soluble polymer is 0.3% by weight in the developing solution in the presence of a non-ionic surface active agent. (for, example, 0.05% by weight Tween20 and 0.3% by weight Triton X-100), a specific increase in sensitivity was observed. When it is used in an amount of 0.6% by weight or 1.5% by weight, the effect was very significant. These results are summarized in Table 1.

TABLE 1

| Water | | Concentration of nuclear | | | | |
|---|---|---|---|---|---|---|
| soluble | Surface active | protein (ng/mL) | | | | |
| polymer | agent | 0 | 5 | 10 | 50 | S/N |
| PVP | Tween 20 | − | + | + | +++ | +++ |
| PVP | None | − | − | ± | ++ | ++ |
| None | Tween 20 | − | − | ± | ++ | ++ |
| None | None | − | − | − | + | + |
| CMC•Na | Tween 20 | ++ | ++ | ++ | +++ | + |
| PVP | Triton x-100 | − | + | + | +++ | +++ |
| PEG | Triton x-100 | ++ | ++ | ++ | +++ | + |
| PVP | Cholic acid Na | − | − | ± | + | + |
| PVP | Tween 20 + | − | + | ++ | +++ | +++ |
| | Triton x-100 | | | | | |

The specific reason for the improvement in signal to noise ratio by a non-ionic surface active agent or a vinyl-based water soluble polymer is not necessarily clear, but the followings can be considered.

When insoluble carriers such as latex particles or colloidal metal particles are used as a labeling substance for the labeling agent in immunochromatography assay, it is known that surface of the particles has a negative charge (for example, see JP-A No. 5-133956). For example, on surface of the colloidal metal particles, anions derived from a reducing agent which is added during production process are adsorbed on the surface and they are maintained in a dispersed state with inhibited agglomeration. It is known that, when a surface active agent at low concentration which does not neutralize the surface charge of the colloidal metal particles is added, several particles are agglomerated with each other in chain form (JP-A No. 2006-58781). The developing solution used in the invention contains a vinyl-based water soluble polymer known as a dispersant for particles as well as a non-ionic surface active agent. It is believed that, due to balancing between their effects, several insoluble carriers that are indirectly captured at a decision part on a chromatography medium are agglomerated, thus resulting in an amplification of positive signal that is observed at the decision part. In particular, for the colloidal metal particles, it is believed that the strength of coloration determined by naked eye is enhanced by the increased number of particles that are accumulated on the decision part due to agglomeration and a change in characteristics of light absorption spectrum of the particles causes a more distinct positive signal at the decision part.

The detection kit of the invention can be used for specifically detecting highly pathogenic avian influenza virus subtype H5N1 when it is included in a sample as a substance of interest. The sample for which the detection kit of the invention can be used is not specifically limited if it is suspected to contain virus subtype H5N1. In mammals such as human, pig, horse, and the like, an airway is usually hit by influenza virus. However, in birds, the infection is observed not only in an airway but also in an intestinal tract (large intestine). As such, preferred examples of the sample include a nasal swab, a nasopharyngeal swab, and an airway swab that are suitable for diagnosis of viral infection in an upper airway and also a cloacal swab and excretes when the sample is taken from a bird. Further, when a test carried out for a dead animal wherein infection with the highly pathogenic avian influenza virus is suspected to be the cause of death, typical organs such as brain, spleen, heart, lung, pancreas, liver, and kidney, drinking water taken by the animal, and the like can also be employed as an appropriate sample in addition to the samples described above. For a sample used for diagnosis of viral infection by detection kit, it is preferably collected within 3 days from the exhibition of clinical symptoms caused by influenza.

When the sample is a liquid, it can be directly applied to a chromatography medium. However, in general, the sample is either suspended or diluted in the developing solution and applied to the chromatography medium.

The method of detecting a substance of interest by using the detection kit of the invention includes the following processes, for example.

As one of the embodiments of the invention, a sample solution containing a substance of interest is mixed in advance with a labeling agent to form a complex of substance of interest

labeling agent in liquid phase, and then it is applied to a chromatography medium. After that, a developing solution is brought into contact with a chromatography medium, together with or subsequent to the sample solution. The developing solution constitutes a mobile phase and it migrates (i.e., develops) with a complex of substance of interest—labeling agent. When the complex of substance of interest—labeling agent migrates over the decision part of the chromatography medium, it is captured by the immobilized first reagent so that the labeling agent indirectly binds to the decision part. Based on the determination of strength of coloration by naked eye, densitometer measurement, or the like, the labeling agent presenting on the decision part can be detected or quantified directly when the labeling substance is an insoluble carrier or by reacting it with a substrate to give a reaction product when the labeling substance is an enzyme.

According to another embodiment of the invention, the labeling agent may be placed on the migration route of the mobile phase in a chromatography medium, i.e., it may be placed in a region between the end part to which the developing solution is applied and the decision part. When the labeling agent is placed on a chromatography medium, it is preferably supported so that it can quickly dissolve in a developing solution and migrates freely via capillary action. At the support part, sugars such as saccharose, sucrose, trehalose, maltose, and lactose, and sugar alcohols such as mannitol may be added for coating, or the support part is coated in advance with them to obtain favorable re-solubility of the reagent. For an application to have the labeling agent coated and dried on a chromatography medium, it can be performed directly on the chromatography medium. Alternatively, it is also possible that the labeling agent is coated and dried on other porous materials like cellulose filter, glass fiber filter, and non-woven nylon fabric to form a labeling agent holding member, which is then applied to get connected via capillaries to the chromatography medium that is immobilized with the first reagent.

As an exemplary detection kit of the invention, the inventors carried out the detection of the highly pathogenic avian influenza virus subtype H5N1 by using monoclonal antibodies 3H4, 3H12, and 4G6 as the first reagent or the second reagent. For the developing solution, the one containing a non-ionic surface active agent (i.e., developing solution A, see Table 5) was used. Naked eye determination was made 15 minutes after the addition of a sample, and the results are given in Tables 2 to 4 below.

TABLE 2
Virus subtype H5N1 (10⁶ pfu/mL)

The first reagent

| | | 3H4 | 3H12 | 4G6 | Pab |
|---|---|---|---|---|---|
| The second | 3H4 | ± | ± | + | ± |
| reagent | 3H12 | ± | ± | + | − |
| | 4G6 | + | + | − | − |
| | Pab | − | − | − | ++ |

TABLE 3
Virus subtype H5N1 (10⁴ pfu/mL)

The first reagent

| | | 3H4 | 3H12 | 4G6 | Pab |
|---|---|---|---|---|---|
| The second | 3H4 | − | − | − | − |
| reagent | 3H12 | − | − | − | − |
| | 4G6 | − | − | − | − |
| | Pab | − | − | − | + |

TABLE 4
Virus subtype H1N1 (10⁶ pfu/mL)

The first reagent

| | | 3H4 | 3H12 | 4G6 | Pab |
|---|---|---|---|---|---|
| The second | 3H4 | − | − | − | − |
| reagent | 3H12 | − | − | − | − |
| | 4G6 | − | − | − | − |
| | Pab | − | − | − | + |

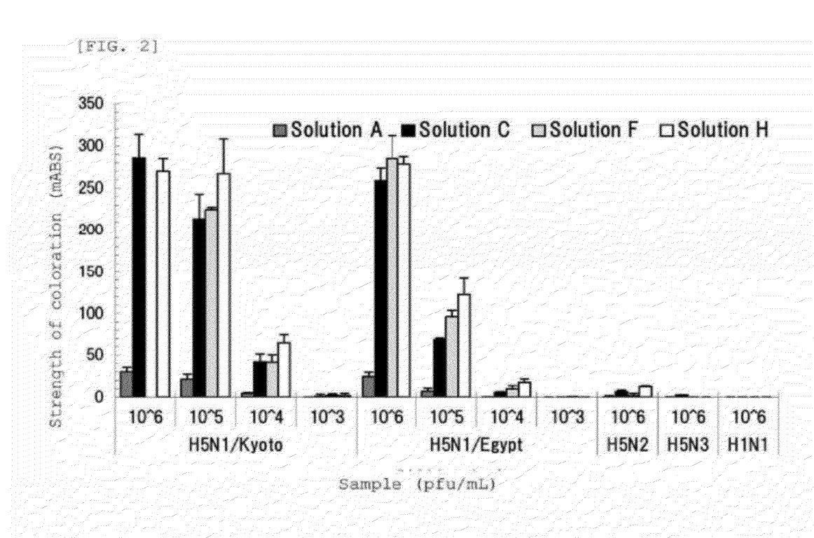

With the detection kit of the invention, the virus subtype H5N1 at the concentration of 10⁶ pfu/mL was detected while the virus subtype H1N1 at the concentration of 10⁶ pfu/mL was not detected. Thus, when an antibody which recognizes the type A influenza virus subtype H5N1 is used as the first or the second reagent, it was possible to detect the virus subtype H5N1. In particular, when the antibody 4G6 is used as the first or the second reagent, significant specificity was shown. The detection kit of the invention showed no cross reactivity for the low pathogenic avian influenza subtypes H5N2 and H5N3 at the concentration of 10⁶ pfu/mL (see, FIG. 2). Meanwhile, as a control for comparison, when a polyclonal antibody (Pab), which has been produced by having AIV H5N1 as an immunogen, was used as the first reagent and the second reagent, not only the virus subtype H5N1 but also the virus subtype H1N1 are detected, yielding no specific detection of AIV H5N1. Thus, by using the detection kit of the invention in which an antibody recognizing specifically the type A influenza virus subtype H5N1 is employed as the first and/or the second reagent, preferably the detection kit of the invention in which the antibody 4G6 is employed as the first and/or the second reagent, specific detection of AIV H5N1 can be achieved.

To improve the measurement sensitivity of the detection kit of the invention, the inventors focused on a developing solution included in the detection kit and studied its composition. As it has been shown that, with the detection kit of the invention, the same specific detection result is obtained even when the measurement subject is switched from AIV H5N1 at the concentration of 10⁶ pfu/mL to the HA recombinant protein of the virus subtype H5N1 (manufactured by ABR) at the concentration of 200 ng/mL, for determination of a developing solution, the HA recombinant protein of the virus subtype H5N1 was used at the concentration of 2 ng/mL (corresponding to 10⁴ pfu/mL of AIV H5N1) as a substance of interest instead of the virus subtype H5N1. Further, as a representative combination of the first reagent and the second reagent, the antibody 4G6 and the antibody 3H4 were selected as the first reagent and the second reagent, respectively.

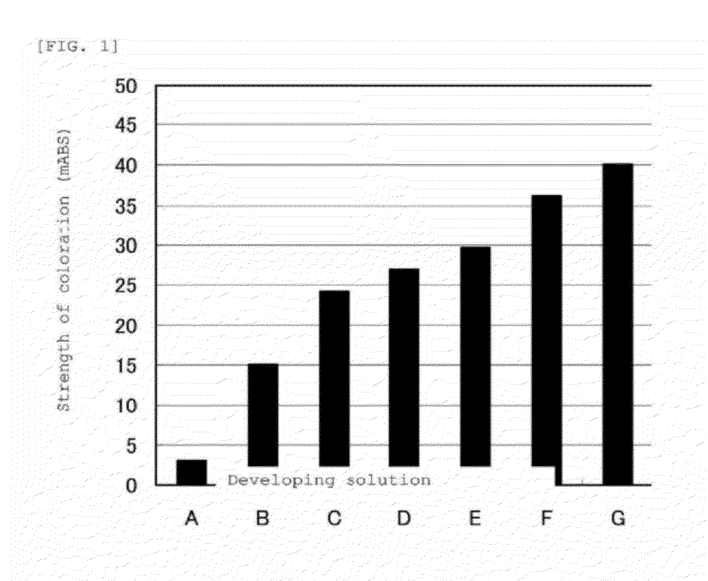

For the determination described above, as a developing solution containing a non-ionic surface active agent, a solution of 120 mM NaCl and 50 mM Tris-HCl added with 0.7% by weight of bovine serum albumin (BSA), 0.3% by weight of Triton X-100, and 0.1% by weight of Tween20 (i.e., developing solution A) was used. As another additive, polyvinyl pyrrolidone was added to the developing solution and examined. The results are shown in Table 5 and FIG. 1.

As it is clearly shown in the results, it was found that by adjusting the developing solution the virus subtype H5N1 can be detected with the sensitivity which corresponds to the measurement sensitivity of a currently commercially available kit for quick diagnosis of human influenza infection, i.e., virus at the concentration of 10⁴ pfu/mL can be determined with naked eye 15 minutes after the addition of a sample. Meanwhile, the virus subtype H5N2 or H5N3 at the concentration of 10⁵ pfu/mL was not detected even when the composition of the developing solution is changed.

TABLE 5

| | | Developing solution | | | | | | | |
|---|---|---|---|---|---|---|---|---|---|
| | | A | B | C | D | E | F | G | H |
| Composition | NaCl | 120 mM | 120 mM | 120 mM | 120 mM | 120 mM | 120 mM | 120 mM | 120 mM |
| | Tris-HCl, | 50 mM | 50 mM | 50 mM | 50 mM | 50 mM | 50 mM | 50 mM | 50 mM |
| | H8.0 | | | | | | | | |
| | BSA | 0.7% | 0.7% | 0.7% | 0.7% | 0.7% | 0.7% | 0.7% | 0.7% |
| | Tritonx-100 | 0.3% | 0.3% | 0.3% | 0.3% | 0.3% | 0.2% | 0.2% | 0.2% |
| | Tween20 | 0.1% | 0.1% | 0.1% | 0.1% | 0.2% | 0.3% | 0.3% | 0.3% |
| | PVP | 0.00% | 0.30% | 0.65% | 0.70% | 0.70% | 0.70% | 0.80% | 0.90% |

| | | | | | | | | |
|---|---|---|---|---|---|---|---|---|
| Strength of | 3.1 | 15.1 | 24.2 | 27.1 | 29.7 | 36.2 | 40.1 | 46.2 |
| coloration | | | | | | | | |

As a result of further adding PVP at the concentration of 0.3% by weight, 0.65% by weight, or 0.7% by weight to a developing solution containing a non-ionic surface active agent, the strength of coloration at the decision part on immunochromatography medium was enhanced in a PVP concentration dependent manner (developing solutions B to D). For the coloration based on the colloidal gold particles that are indirectly attached on the decision part, naked eye determination and measurement by Immunochromato Reader (trade name, manufactured by Hamamatsu Photonics K.K.) were carried out. When the measurement value obtained by Immunochromato Reader (trade name, manufactured by Hamamatsu Photonics K.K.) is 20.0 or more, coloration can be clearly identified with naked eye. By further adding PVP to a developing solution containing a non-ionic surface active agent, the strength of coloration was enhanced. Further, when PVP is added at the concentration of 0.65% by weight or more, more distinct positive reaction was obtained. Next, it was examined whether or not the effect of enhancing the coloration strength by addition of PVP is affected by the concentration of a non-ionic surface active agent. Specifically, when the concentration of a non-ionic surface active agent is increased in the presence of 0.7% by weight PVP, further enhancement in coloration strength was shown (developing solutions D to F). In addition, when the PVP concentration was increased from 0.7% by weight to 0.9% by weight in the presence of 0.3% by weight Tween20 and 0.2% by weight Triton X-100, the strength of coloration at the decision part was further enhanced (developing solutions F to H). Thus, it was found that the strength of coloration at the decision part is enhanced by adding a non-ionic surface active agent and PVP to the developing solution of the invention. This effect is in match with the results determined in detail in the specification of Japanese Patent Application No. 2008-182630.

As it is evident that high sensitivity can be obtained for the detection kit of the invention by modifying the composition of a developing solution, by actually using a developing solution added with a non-ionic surface active agent and PVP, measurement of avian influenza virus was carried out. In addition to the strain A/crow/Kyoto/53/2004 (H5N1) as the virus subtype H5N1 that is used for preparation of an antibody, the strain A/chicken/Egypt/CL-61/2007 (H5N1) which has been isolated from a chicken in Egypt in 2007 was used. As a control, the virus subtype H1N1 isolated from human (A/Puertorico/8/34), the virus subtype H5N2 (A/duck/HongKong/342/78) and virus subtype H5N3 (A/duck/HongKong/820/80) isolated from duck were used. Naked eye determination and measurement by Immunochromato Reader (trade name, manufactured by Tanaka Kikinzoku Kogyo K.K.) were carried out for the coloration at the decision part. When the measurement value obtained by Immunochromato Reader (trade name, manufactured by Tanaka Kikinzoku Kogyo K.K.) is 15.0 or more, coloration can be clearly identified with naked eye. The strength of coloration that is obtained from the measurements of the type A influenza virus using various developing solution is given in Table 6 and FIG. 2.

TABLE 6

| | | | Developing solution | | | |
|---|---|---|---|---|---|---|
| | | | A | C | F | H |
| Sample | H5N1/Kyoto | 10\hat{ }6 | 30.2 | 285.7 | 334.0 | 270.0 |
| (pfu/mL) | | 10\hat{ }5 | 21.5 | 214.0 | 225.0 | 267.3 |
| | | 10\hat{ }4 | 4.5 | 42.0 | 41.7 | 64.3 |
| | | 10\hat{ }3 | 0.0 | 1.3 | 2.0 | 1.7 |
| | H5N1/Egypt | 10\hat{ }6 | 24.5 | 259.0 | 285.0 | 278.7 |
| | | 10\hat{ }5 | 7.2 | 69.0 | 96.0 | 122.3 |
| | | 10\hat{ }4 | 0.2 | 6.0 | 10.0 | 17.7 |
| | | 10\hat{ }3 | 0.0 | 0.0 | 0.3 | 0.0 |
| | H5N2 | 10\hat{ }6 | 0.5 | 7.0 | 1.7 | 10.0 |
| | H5N3 | 10\hat{ }6 | 0.0 | 2.3 | 0.0 | 0.0 |
| | H1N1 | 10\hat{ }6 | 0.0 | 0.0 | 0.0 | 0.0 |

As a result of using a developing solution added with a non-ionic surface active agent and PVP, the virus subtype H5N1 can be detected at the target concentration of 10⁴ pfu/mL (intensity≧15, naked determination +). Meanwhile, no coloration at the decision part was observed with naked eye for the virus subtype H5N2 and the virus subtype H5N3 even when they were determined at a higher concentration of 10⁶ pfu/mL. By adding a non-ionic surface active agent and PVP to a developing solution, the strength of coloration at a decision part was enhanced for a positive sample containing AIV H5N1 as a substance of interest (i.e., signal), while it was inhibited for a negative sample not containing AIV H5N1 as a substance of interest (i.e., noise). Among the additives added to the developing solution for immunochromatography assay, several additives are known as a sensitizer which has an effect of increasing signal strength. However, as they simultaneously increase the noise strength, many of them yield a pseudo-positive reaction when a negative sample is measured. However, when a developing solution containing a non-ionic surface active agent and PVP is used, the signal strength is increased, and therefore not only the virus subtype H5N1 at low concentration can be measured with high sensitivity but also the generation of the noise due to viruses other than the subtype H5N1 (i.e., the virus subtype H5N2 or virus subtype H5N3) is inhibited. Accordingly, the virus subtype H5N1 can be specifically detected with high sensitivity. This effect of enhancing the measurement sensitivity by a developing solution was also shown even when the antibody 3H12 was used as the second reagent. Furthermore, the same effect was obtained when the monoclonal antibodies used for the first reagent and the second reagent are switched each other.

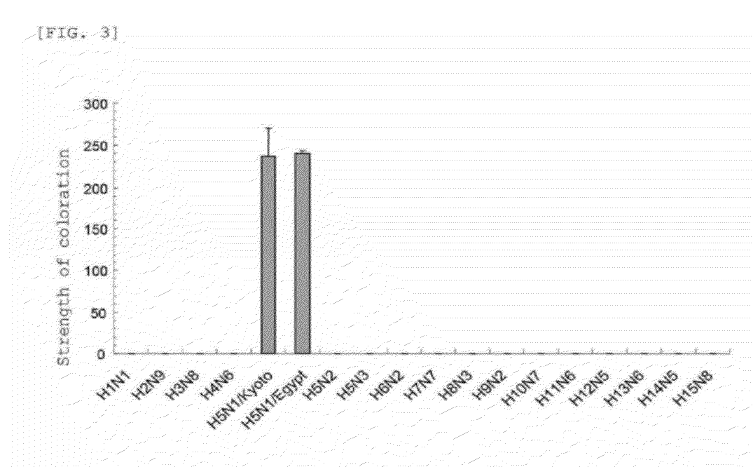

Furthermore, in order to confirm more clearly whether the detection kit of the invention can detect the virus subtype H5N1 only, cross reactivity test was carried out by using influenza virus strains having various hemagglutinin (HA) subtype and neuraminidase (NA) subtype. The results are given in Table 7 and FIG. 3.

TABLE 7

| Influenza virus strain | Naked eye |
|---|---|
| A/Puertorico/8/34(H1N1) | − |
| A/Duck/HongKong/278/78(H2N9) | − |
| A/duck/Ukrine/1/63(H3N8) | − |
| A/duck/Czechslovakia/1/56(H4N6) | − |
| A/crow/Kyoto/53/2004 (H5N1) | + |
| A/chicken/Egypt/CL-61/2007(H5N1) | + |
| A/duck/HongKong/342/78 (H5N2) | − |
| A/duck/HongKong/820/80 (H5N3) | − |
| A/turkey/Massachusets/3470/65 (H6N2) | − |
| A/wigeon/osaka/1/2001 (H7N7) | − |
| A/turkey/Ontario/6118/68 (H8N3) | − |
| A/turkey/Wisconsin/1/66 (H9N2) | − |
| A/chicken/Germany/N/49 (H10N7) | − |
| A/duck/England/1/56 (H11N6) | − |
| A/duck/Alberta/60/76 (H12N5) | − |
| A/gull/Maryland/704/77 (H13N6) | − |
| A/mallard/Astrakhan/263/82 (H14N5) | − |
| A/duck/Australia/341/83 (H15N8) | − |

As described above, with the detection kit of the invention, influenza virus of the virus subtype H5N1 can be detected with high sensitivity while viruses other than the virus subtype H5N1 cannot be detected at all with naked eye. In other words, it is evident that the detection kit of the invention can be used for detection of the influenza virus subtype H5N1 with high specificity.

Herein below, the invention is explained in greater detail with reference to the Examples. However, the invention is not limited by the Examples.

### EXAMPLES

### Example 1

### 1. Production of a Decision Part on Chromatography Medium

On a 25×2.5 cm nitrocellulose membrane (trade name; HF 120, manufactured by Millipore K.K.), any one of the monoclonal antibodies 3H4, 3H12, and 4G6 (i.e., the first reagent) against the highly pathogenic influenza virus A (H5N1), which has been diluted with phosphate buffer (pH 7.4) containing 5% by weight of isopropyl alcohol, was coated using an antibody coater (manufactured by BioDot Inc.) to have the concentration of 1.3 mg/mL followed by drying at 42° C. for 60 minutes to produce a decision part on a chromatography medium.

### 2. Production of Labeling Agent Solution

To 0.5 mL of colloidal gold suspension (manufactured by Tanaka Kikinzoku Kogyo K.K., with the average particle diameter of 60 nm), 0.1 mL of 50 mM phosphate buffer (pH 7.4) was added and mixed. Then, 0.1 mL of any one of the monoclonal antibodies 3H4, 3H12, and 4G6 (i.e., the second reagent) against the highly pathogenic influenza virus A (H5N1), which has been diluted with 5 mM phosphate buffer (pH 7.4), was added and the resultant mixture was left to stand for 10 minutes at room temperature. Subsequently, 0.1 mL of 10% by weight bovine albumin serum (BSA) diluted with 10 mM phosphate buffer was added and fully stirred, followed by centrifuge for 15 minutes at 8000×g. The supernatant was removed and added with 1 mL of 10 mM phosphate buffer (pH 7.4). By using an ultrasonicator, the colloidal labeling agent was dispersed well and subjected to centrifuge for 15 minutes at 8000×g. The supernatant was removed, added with the phosphate buffer described above, and dispersed well using an ultrasonicator to obtain a solution of labeling agent.

### 3. Production of Chromatography Medium

The solution of labeling agent produced above was evenly applied to a 16×100 mm glass fiber pad (trade name: GFCP203000, manufactured by Millipore K.K.) and then dried in a vacuum drier to give a labeling agent holding member. Subsequently, on a substrate consisting of a backing sheet, the nitrocellulose membrane in which the decision part is formed as described above, the labeling agent holding member, a glass fiber sample pad to be used as a sample addition part (8000006801, manufactured by Pall Corporation), and an absorption pad for absorbing a developed sample or labeling agent were attached. Finally, the resultant was cut using a cutter to have the width of 5 mm, and therefore a chromatography medium was produced.

### 4. Measurement

By using the chromatography medium produced in section 3 above, presence or absence of the highly pathogenic influenza virus A/crow/Kyoto/53 (H5N1) was determined with a sample containing the virus as a substance of interest. Specifically, a 50 mM Tris-hydrochloride buffer solution (pH 8.0) containing 0.3% by weight of Triton X-100 (trade name, HLB value: 13.5), 0.1% by weight of Tween20 (trade name, HLB value: 16.7), 0.7% by weight of bovine serum albumin, and 120 mM sodium chloride was employed as a developing solution (i.e., developing solution A, see Table 5), and a solution (120 μL) obtained by adding various influenza viruses which have been diluted in 10 mM phosphate buffered physiological saline (pH 7.4) to the developing solution was used as a test sample. By having the highly pathogenic influenza virus A/crow/Kyoto/53 (H5N1) at the concentration of 10⁶ pfu/mL (Table 2) or 10⁴ pfu/mL (Table 3) as a positive sample and the influenza virus A/Puertorico/8/34 (H1N1) at the concentration of 10⁶ pfu/mL (Table 4) as a negative sample, the samples were applied on the sample pad of the chromatography medium, and then developed. Fifteen minutes later, naked eye determination was made. The sample showing identifiable redline at the test line of the decision part was decided as “+”, while the sample showing identifiable redline but very weak red line was decided as “±”, and the sample not showing any identifiable red line was decided as “−”. The results are given in Tables 2 to 4.

### Comparative Example 1

The measurement was carried out in the same manner as Example 1 except that the polyclonal antibody against highly pathogenic influenza virus A (H5N1) is used for both the first reagent and the second reagent. The results are given in Tables 2 to 4.

### Example 2

The measurement was carried out in the same manner as Example 1 except that the antibody 4G6 and the antibody 3H4 are used as the first reagent and the second reagent, respectively, and strength of coloration is measured by Immunochromato Reader (trade name, manufactured by Hamamatsu Photonics K.K.) using the developing solution with various compositions as described in Table 5 and 2 ng/mL of H5N1 HA recombinant protein (manufactured by ABR) as a substance of interest. The results are given in Table 5 and FIG. 1.

### Example 3

The measurement was carried out in the same manner as Example 1 except that the antibody 4G6 and the antibody 3H4 are used as the first reagent and the second reagent, respectively, and strength of coloration is measured by Immunochromato Reader (trade name, manufactured by Tanaka Kikinzoku Kogyo K.K.) using the developing solution A, C, F, and H described in Table 5 and various influenza viruses as a substance of interest, i.e., 10⁶ pfu/mL, 10⁵ pfu/mL, 10⁴ pfu/mL, or 10³ pfu/mL of the highly pathogenic influenza virus A/crow/Kyoto/53/2004 (H5N1), 10⁶ pfu/mL, 10⁵ pfu/mL, 10⁴ pfu/mL, or 10³ pfu/mL of the highly pathogenic influenza virus A/chicken/Egypt/CL-61/2007 (H5N1), 10⁶ pfu/mL of the influenza virus A/duck/HongKong/342/78 (H5N2), 10⁶ pfu/mL of the influenza virus A/duck/HongKong/820/80 (H5N3), and influenza virus A/Puertorico/8/34 (H1N1). The results are given in Table 6 and FIG. 2.

### Example 4

The measurement was carried out in the same manner as Example 1 except that the antibody 4G6 and the antibody 3H4 are used as the first reagent and the second reagent, respectively, and strength of coloration is measured by Immunochromato Reader (trade name, manufactured by Tanaka Kikinzoku Kogyo K.K.) together with naked eye determination using the developing solution H described in Table 5 and 10⁵ pfu/mL of the various influenza various described in Table 7. The results are given in Table 7 and FIG. 3.

### INDUSTRIAL APPLICABILITY

The detection kit of the invention has a detection sensitivity that is practically usable and can be used for specific detection of highly pathogenic avian influenza virus subtype H5N1. Therefore, it has an industrial applicability in that it can be used for rapid and convenient test of influenza infection caused by virus subtype H5N1.

1. A detection kit for type A influenza virus subtype H5N1, for detecting a substance of interest in sample by immunochromatography assay, including a chromatography medium containing a first reagent at decision part, a labeling agent in which a second reagent is conjugated to a labeling substance, and a developing solution; comprising one or both of the first reagent and the second reagent being an antibody specifically recognizing type A influenza virus subtype H5N1.

2. The detection kit according to claim 1, wherein the first reagent specifically recognizes type A influenza virus subtype H5N1.

3. The detection kit according to claim 1, wherein the antibody specifically recognizing type A influenza virus subtype H5N1 is a monoclonal antibody recognizing a conformational epitope containing an asparaginic acid that is the 59^{th} amino acid of hemagglutinin in the virus subtype H5N1.

4. The detection kit according to claim 3, wherein the monoclonal antibody recognizing the conformational epitope containing an asparaginic acid that is the 59^{th} amino acid of hemagglutinin in the virus subtype H5N1, is a monoclonal antibody produced by a mouse-mouse hybridoma 4G6 (deposit number: FERM BP-11130).

5. The detection kit according to claim 1, wherein the remaining first reagent or the second reagent is a monoclonal antibody recognizing a consecutive epitope presenting in the region of 273-342aa in hemagglutinin HA1 domain of influenza virus subtype H5N1.

6. The detection kit according to claim 5, wherein the monoclonal antibody recognizing a consecutive epitope presenting in the region of 273-342aa in hemagglutinin HA1 domain of influenza virus subtype H5N1 is a monoclonal antibody produced by a mouse-mouse hybridoma 3H4 (deposit number: FERM BP-11173) or a mouse-mouse hybridoma 3H12 (deposit number: FERM BP-11174).

7. The detection kit according to claim 1, wherein the developing solution contains a non-ionic surface active agent with HLB value of from 13 to 18.

8. The detection kit according to claim 7, wherein a concentration of the non-ionic surface active agent is 0.1 to 1.0%.

9. The detection kit according to claim 1, wherein the developing solution further contains a vinyl-based water soluble polymer having a polar group with an oxygen atom and a nitrogen atom.

10. The detection kit according to claim 9, wherein a concentration of the vinyl-based water soluble polymer is 0.5 to 2.0%.

11. The detection kit according to claim 9, wherein the vinyl-based water soluble polymer is polyvinyl pyrrolidone.

12. The detection kit according to claim 7, wherein the labeling substance is an insoluble carrier.

13. The detection kit according to claim 12, wherein the insoluble carrier is a colloidal gold particle.

14. A method of detecting type A influenza virus subtype H5N1 in a sample by using the detection kit according to claim 1, comprising processes of bringing a sample into contact with a chromatography medium, bringing a labeling agent into contact with the chromatography medium together with or subsequent to the sample, and developing the sample and the labeling agent with an aid of a developing solution.
